(19) United States
(12) Patent Application Publication
(10) Pub. No.: US 2003/0211132 A1
(43) Pub. Date: Nov. 13, 2003

(54) Method for reducing radioactivity in the human body

(76) Inventor: Tihiro Ohkawa, La Jolla, CA (US)

Correspondence Address: NEIL K. NYDEGGER, NYDEGGER & ASSOCIATES, 348 OLIVE STREET, SAN DIEGO, CA, 92103, US

(21) Appl. No.: 10/144,011

(22) Filed: May 10, 2002

(51) Int. Cl. A61K 47/00; A61K 33/00

(52) U.S. Cl. 424/439; 424/717

(57) ABSTRACT

Food products and methods for synthesizing food products for reducing naturally occurring radioactive potassium isotope (⁴⁰K) in the human body requires separating the radioactive potassium isotope (⁴⁰K) from the non-radioactive isotopes (i.e. ³⁹K) in potassium. Various food products can then be prepared using the ³⁹K. These food products include manufactured products, such as sports drinks, baking soda, and dietary tablets, as well as altered liquid food products, such as orange juice and tomato juice.

### FIELD OF THE INVENTION

The present invention pertains generally to methods for reducing a person's exposure to radiation. More particularly, the present invention pertains to methods and products for reducing radioactive nuclides that are ingested or otherwise introduced into the human body. The present invention is particularly but not exclusively useful as a method for reducing radiation exposure from naturally occurring radioactive potassium isotopes (⁴⁰K) inside the body.

### BACKGROUND OF THE INVENTION

In our daily lives, we are each exposed to various types of naturally occurring ionizing radiation which is commonly referred to as background radiation. Naturally occurring background radiation comes from a number of sources that include terrestrial radiation, inhaled radionuclides, cosmic radiation and internal radionuclides. It happens, however, that some naturally occurring radioactive elements find their way into our bodies. Chief among these is the radionuclide potassium-40 (⁴⁰K).

It is well known that potassium is an essential element for human physiology. In general, the body of an average adult contains about 250 grams of potassium and the daily dietary requirement is about 2 to 5 grams.

Naturally occurring potassium, such as that which is ingested by humans with food products, consists of three separate isotopes: ³⁹K (93.1%), ⁴⁰K (0.0118%) and ⁴¹K (6.88%). Of these, only the ⁴⁰K isotope is radioactive. Specifically, the radioactivity of ⁴⁰K involves β⁻-decay (89.4%) at 1.31 MeV, K-capture (10.6%) with γ at 1.46 MeV, and β⁺-decay (0.001%) at 1.5 MeV. The half-life of ⁴⁰K is 1.28×10⁹ years. Fortunately, only a small fraction of all potassium (i.e. about 0.0118%) is in the form of the radioactive isotope of potassium (⁴⁰K).

With the above in mind, the bodily inventory of 250 grams of naturally occurring potassium for the typical, average adult corresponds to approximately 4.5×10²⁰ nuclei of ⁴⁰K. The decay time constant of this ⁴⁰K is 5.8×10¹⁶ seconds, and therefore the corresponding activity is 7.8×10³ decays per second. This then corresponds to about 0.21 microcuries (one microcurie equals thirty-seven thousand disintegrations every second). Stated differently, and using another typical measurement, the radioactivity from ⁴⁰K that originates inside the body causes an exposure of about 0.47 millisieverts per year.

Radioactive isotopes that are ingested or inhaled, and that therefore result in ionizing radiation emitted from inside the body, are not shielded and are the most damaging. On the other hand, radiation that originates outside the body may be stopped by clothing or by the skin if the energy level is not too high. Nevertheless, even when very high energy radiation originates outside the body, and penetrates the skin, it causes less ionization and less cell damage to vital organs than does comparable radiation which originates inside the body. All of the radiation exposure due to ⁴⁰K is, unfortunately, from ingested or internal radionuclides.

For a rough approximation of the ionizations that are caused inside the body due to ⁴⁰K, it can be assumed that the ⁴⁰K decay is all due to β decay. Accordingly, for purposes of this approximation, β⁺-decay and γ radiation are ignored. With this in mind, the average electron energy for β-decay is 0.44 MeV, or about one-third of the decay energy. It is also assumed that the penetrating range of the electrons through body tissues is approximately equivalent to their penetration range in water. For electrons at 0.44 MeV the penetration range is 0.22 cm. The average energy required for ionization is 35 eV. Therefore, β-decay electrons at 0.44 MeV produce about 5.7×10⁴ ion pairs per cm (i.e. 5.7×10⁴ ionizations per cm).

To approximate the extent that ⁴⁰K irradiation adversely affects human cells, the average size of a human cell is estimated to have an average dimension of approximately 30 microns. Therefore, using the penetration range approximated above (i.e. 0.22 cm), radiation may pass through as many as 73 cells. The number of the ionizations per cell is 170 (i.e. 5.7×10⁴ ionizations/cm times 30×10⁻⁴ cm/cell). The decay rate of 0.21 microcuries corresponds to 5.7×10⁵ cells irradiated every second. Thus, if the volume of the human body is estimated at 0.06 m³, the total number of cells in the body is approximately 2.2×10¹². Consequently, for this approximation, the time interval between successive ionizations of a cell is approximately 3.9×10⁶ seconds. Stated differently, on average, each cell will be ionized once every 45 days.

These ionizations of human cells can potentially lead to problems. As is well known, irradiation of cells causes damage that may eventually lead to cell mutations. If mutated cells do not die, but rather undergo cell division, their daughters may then contain the same mutations as the parent cells. For a worst case scenario wherein the cell division of mutant daughters leads to cancerous cells, a reduction of the internal irradiation rate would logically reduce the probability of the occurrence of cancer. Although the human body has evolved over the ages in an environment that includes potassium-40 and other potential carcinogens, it seems prudent to reduce the body's exposure to potentially cancerous matter.

In light of the above, it is an object of the present invention to provide the capability to reduce radioactive isotope ⁴⁰K from the dietary intake of potassium. It is a further object of the present invention to provide a process and method for synthesizing a food product that is a source of nutrient potassium and that is largely free of ionizing radioactive ⁴⁰K. Another object of the present invention is to provide a process for removing ⁴⁰K from liquid food products that are a source of nutrient potassium. Still another object of the present invention is to provide a method to deplete naturally occurring ⁴⁰K in the human body that is simple to implement and that is cost effective.

### SUMMARY OF THE PRFERRED EMBODIMENTS

As stated above, the present invention is directed to food products and methods for synthesizing food products which can be ingested into the body. Specifically, the food products that pertain to the present invention contain nutrient potassium (K), but they are substantially free of the radioactive potassium isotope (⁴⁰K). This requires separating radioactive isotopes (⁴⁰K) from the non-radioactive isotopes (³⁹K, ⁴¹K) that are in potassium (K). Since ⁴¹K is non-radioactive and a very minor constituent of potassium, the object here is to recover a quantity of substantially pure non-radioactive isotope (³⁹K). Food products and tablets can then be prepared using the recovered quantity of non-radioactive isotope (³⁹K) to provide a source of nutrient potassium that is substantially free of the radioactive isotope ⁴⁰K.

By ingesting the food products, or tablets, that are synthesized by the methods of the present invention, the body receives a source of nutrient potassium (K) which does not add to the body's inventory of radioactive potassium isotope (⁴⁰K). Thus, by supplementing the body's normal intake of potassium (K) with a potassium source having very low ⁴⁰K, or non-existent ⁴⁰K, the body is able to naturally deplete ⁴⁰K through the normal bodily processes. Examples of the food products that can be effective for this purpose include liquid products, sports drinks, mineral tablets and baking soda.

An additional aspect of the present invention is directed to processes that involve liquid food products which have substantial quantities of natural nutrient potassium (K) such as orange juice or tomato juice. Specifically, as envisioned for the present invention, the radioactive potassium isotope (⁴⁰K) in these natural liquid food products may be removed by dialysis. Dialysis, however, will remove all potassium (K) from these liquid products. Thus, as envisioned for the present invention, a quantity of substantially pure non-radioactive potassium isotopes (³⁹K, ⁴¹K) can then be added back into the product to prepare a product that is substantially free of radioactive potassium isotope (⁴⁰K).

### DESCRIPTION OF THE PREFERRED EMBODIMENTS

The present invention is directed generally to ingestible products, and to methods for the preparation of these products. Specifically, the present invention is directed to products that, when ingested, will help supply a person's daily requirement for potassium. Importantly, the potassium that is in the products of the present invention is altered by isotope separation methods so as to be substantially free of the radioactive potassium isotope ⁴⁰K.

From the table of exemplary products that is presented below, it can be seen that various foods and drinks are excellent sources for dietary potassium. Notably, dairy products, vegetables and fruits are examples of natural food items that are rich in natural dietary potassium. As discussed above, however, the natural potassium in these food items includes the radioactive potassium isotope ⁴⁰K. As also discussed above, for health reasons, it is desirable to reduce the amount of ⁴⁰K inside the body of an individual.

| Food Item | Quantity (grams) | K Content (milligrams) |
|---|---|---|
| Beer | 360 | 115 |
| Coffee | 180 | 124 |
| Orange Juice | 248 | 496 |
| Avocado | 173 | 1,097 |
| Peanuts | 145 | 1,018 |
| Red Wine | 102 | 113 |
| Milk | 244 | 370 |
| Peach | 87 | 171 |
| White Bread | 454 | 608 |
| Beef | 65 | 306 |
| Tomato Juice | 244 | 537 |
| Spinach | 55 | 307 |
| Gatorade ® | 240 | 30 |
| Baking Powder | 4.3 (1 tsp) | 892 |
| Mineral Tablet | 1.2 (1 tab) | 25 |

In general, it is basically impractical to remove the radioactive potassium isotope ⁴⁰K from many natural food items (e.g. avocados or peanuts). Accordingly, the present invention is not directly concerned with the direct alteration of such natural foods. Instead, the concern here is for the preparation of tablets and food products that can be synthesized using a potassium additive which is substantially devoid of the radioactive potassium isotope ⁴⁰K.

For the synthesis or manufacture of products in accordance with the present invention, it is first necessary to prepare a potassium additive that is substantially free of the radioactive potassium isotope ⁴⁰K. This can be accomplished in any of several ways, all of which are known in the pertinent art. For example, it is known that plasma centrifuges, gas diffusion devices, laser procedures and chemical processes may be variously employed to separate isotopes from each other, such as to remove radioactive potassium isotopes ⁴⁰K directly from potassium. In any event, for the present invention it is desirable that the resultant potassium additive be essentially pure ³⁹K potassium.

Because the potassium isotopes ⁴⁰K and ³⁹K are chemically equivalent, it happens that the absence of ⁴⁰K potassium in food products is unnoticeable. Stated differently, a food product containing only ³⁹K potassium will have the same basic taste and consistency characteristics as does naturally occurring potassium, before the ⁴⁰K isotope was removed. With this in mind, the use of the ³⁹K potassium in accordance with the present invention should be considered in two contexts. First, its use in the preparation of chemical compounds (e.g. baking soda), or tablets for use as dietary supplements. Second, its use as a replenishment for the natural potassium that has been removed from otherwise natural food items (e.g. orange juice).

When ³⁹K potassium is to be used as a replenishment for natural food items (e.g. orange juice), it is first necessary to remove the natural potassium from the food item. As envisioned for the present invention, this can be done by conventional dialysis techniques. As is well known, dialysis results in the separation of colloids and crystalloids in solution by the differences of their rates of diffusion through a semipermeable membrane. Thus, dialysis removes all small ions such as salts or acids with small molecular weights. ³⁹K potassium, which has been prepared as disclosed above, can then be used to replenish the ⁴⁰K and other small molecular weight constituents that were removed from the solution. Orange juice, of course, is not the only example of a food item that can be altered to remove the radioactive isotope ⁴⁰K potassium. Other liquids and juices that are high in potassium, such as milk or tomato juice, can also be altered in this same manner. On the other hand, for manufactured drinks, such as sport drinks, ³⁹K potassium can be used directly as a constituent during manufacture.

Regardless whether ³⁹K is used as a replenishment or as a constituent of manufacture, its use is beneficial in reducing the amount of ⁴⁰K potassium that is ingested by an individual. Specifically, in each instance, the portion of an individual's daily potassium intake that is supplied by products or food items containing pure ³⁹K potassium will reduce the individual's exposure to a proportional quantity of the radioactive isotope ⁴⁰K.

While the particular Method For Reducing Radioactivity in the Human Body as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A process for synthesizing a food product for ingestion into the body, containing nutrient potassium (K), with reduced quantities of radioactive isotope (⁴⁰K), which comprises the steps of:
separating radioactive isotopes (⁴⁰K) from non-radioactive isotopes (³⁹K, ⁴¹K) in potassium (K) to recover a quantity of substantially pure non-radioactive isotope (³⁹K); and
preparing the food product using said quantity of non-radioactive isotope (³⁹K) to provide a source of nutrient potassium substantially free of the radioactive isotope ⁴⁰K to facilitate natural depletion of ⁴⁰K within the body.

2. A process as recited in claim 1 wherein the food product is a liquid.

3. A process as recited in claim 1 wherein the food product is a tablet.

4. A process as recited in claim 1 wherein the food product is baking soda.

5. A food product for ingesting in the human body for providing a source of potassium nutrient (K) comprising a quantity of substantially pure non-radioactive isotopes (³⁹K, ⁴¹K) and thereby allowing the body to naturally deplete radioactive isotope (⁴⁰K) from the body.

6. A food product as recited in claim 5 wherein the food product is a drink.

7. A food product as recited in claim 5 wherein the food product is a tablet.

8. A food product as recited in claim 5 wherein the food product is baking soda.

9. A process for synthesizing a liquid food product containing nutrient potassium (K) which comprises the steps of:
separating radioactive isotopes (⁴⁰K) from non-radioactive isotopes (³⁹K, ⁴¹K) in potassium (K) to recover a quantity of substantially pure non-radioactive isotope (³⁹K);
dialyzing the liquid in order to remove the potassium (K) therefrom; and
adding the quantity of substantially pure non-radioactive potassium isotopes (³⁹K, ⁴¹K) to prepare a product that is substantially free of radioactive potassium isotope (⁴⁰K).

10. A process as recited in claim 9 wherein the liquid food product is orange juice.

11. A process as recited in claim 9 wherein the liquid food product is tomato juice.

12. A process as recited in claim 9 wherein the liquid food product is milk.
